(19) United States
(12) Patent Application Publication
(10) Pub. No.: US 2025/0179300 A1
(43) Pub. Date: Jun. 5, 2025

(54) NATURAL SODIUM BENTONITE CLAY WITH IMPROVED RHEOLOGICAL PROPERTIES

(71) Applicant: BYK-CHEMIE GMBH, Wesel (DE)

(72) Inventors: KLAUS DZIWOK, FREILASSING (DE); ANETTE LORK, REES (DE); SYLVIA BUHNE, DUISBURG (DE); AXEL WOOCKER, REES (DE); HANNELORE KLEEBERGER, LANGENPREISING (DE); WOLFGANG HEININGER, MOOSBURG (DE)

(21) Appl. No.: 18/842,606

(22) PCT Filed: Apr. 3, 2023
(86) PCT No.: PCT/EP2023/058709

§ 371 Date: Aug. 29, 2024

(30) Foreign Application Priority Data
Apr. 6, 2022 (EP) 22167038.3

(51) Int. Cl. C09C 1/30; C09D 7/43; C09D 7/61

(52) U.S. Cl. CPC C09C 1/3027; C09D 7/43; C09D 7/61; C01P 2004/61; C01P 2006/22; C01P 2006/82

(57) ABSTRACT

The invention relates to a process for treatment of a natural clay material comprising sodium bentonite comprising i) Preparing an aqueous slurry of a natural clay material comprising sodium bentonite, ii) Removing non-sodium bentonite impurities from the aqueous slurry, and iii) Removing water from the aqueous slurry by spray drying in a spray drying apparatus to prepare a solid treated sodium bentonite clay.

The invention relates to process for treatment of a natural clay material comprising sodium bentonite, to a treated natural sodium bentonite clay, which is obtainable by the process, to the use of the treated natural sodium bentonite clay, and to a method of controlling the rheology of an aqueous composition.

The different types of bentonite are each named after the respective dominant cation. For industrial purposes, two main classes of bentonite are recognized: sodium and calcium bentonite. Sodium bentonite is the more valuable, but calcium bentonite is more common. Natural occurring sodium bentonites are typically not available in high purities. Typically, they carry significant amounts of inert minerals as impurities in the mineral, which detracts from their use as rheological additives.

Natural calcium bentonite may be converted to sodium bentonite, termed sodium beneficiation or sodium activation, to exhibit many of sodium bentonite's properties by an ion exchange process. As commonly practiced, this means adding 5-10% of a soluble sodium salt, such as sodium carbonate, to wet calcium bentonite, mixing well, and allowing time for the ion exchange to take place and water to remove the exchanged calcium. Some properties, such as viscosity and fluid loss of suspensions, of sodium-beneficiated calcium bentonite are not fully equivalent to those of natural sodium bentonite. For example, residual calcium carbonates, formed if exchanged cations are insufficiently removed, may result in inferior performance of the artificial sodium bentonite.

CN 111269606 A describes a process of modifying of a calcium bentonite powder to a sodium bentonite powder. The process comprises treatment of the calcium bentonite with a source of sodium cations in the presence of water, preparation of an aqueous slurry, followed by centrifugation, pH adjustment, and drying.

CN 102283860 A relates to a preparation method of a montmorillonite preparation, which belongs to the technical field of medicines. The examples describe the treatment of bentonite with water, removal of sand, and spray drying the resulting suspension.

There is an ongoing need for providing bentonite clays with improved rheological properties, in particular bentonites showing improved thickening efficiency in water-based formulations. Furthermore, it is highly desirable that the clays can be dispersed in water or aqueous formulations easily and quickly, with reduced need for application of shear force.

The invention provides a process for treatment of a natural clay material comprising sodium bentonite comprising
i) Preparing an aqueous slurry of a natural clay material comprising sodium bentonite,
ii) Removing non-sodium bentonite impurities from the aqueous slurry, and
iii) Removing water from the aqueous slurry by spray drying in a spray drying apparatus to prepare a solid treated sodium bentonite clay,
wherein the solid treated bentonite clay has a content of exchangeable sodium ions in an amount of 100 mmol/100 g or less, and a content of exchangeable calcium ions in an amount of 18 mmol/100 g or less, calculated on the dry weight of the clay.

The process of the invention provides a treated bentonite clay having improved thickening efficiency in water-based formulations. Furthermore, the treated clays can be dispersed in water or aqueous formulations easily and quickly, with reduced need for application of shear force.

The starting material used in the process of the invention is a natural clay material comprising sodium bentonite. Natural clay materials are materials which are obtained from clay mines and which have not been treated or modified, other than by physical methods, such as grinding or sieving to obtain a desired particle size.

Bentonites are natural clay minerals, wherein the major component is montmorillonite. Generally, bentonites contain montmorillonite in the range of 30 to 90% by weight. The montmorillonites present in bentonites are platelet shaped aluminum-silicates which are stacked on each other. The platelets typically are slightly negatively charged. Therefore, they carry cations in the interlayer between the platelets in order to compensate the negative charges of the layers. Their applications are typically due to their high surface area and platelet like structure which gives them specific advantage in gelling water or solvents or adsorbing specific substances or providing barrier properties. In most of these applications a separation of the platelets into single or small stack platelets is required in order to achieve the best properties. The bentonites can provide this swelling into platelets in case the interlayer cations are single charged, especially if the interlayer cations are sodium or lithium. Bentonite clays which carry mostly divalent cations between their layers a Ca and Mg ions are only slightly swellable and the single platelets of aluminum-silicate cannot be removed from each other in water completely.

The natural clay material comprising sodium bentonite used as raw material for the present invention generally has a certain content of sodium ions. The content of sodium ions is suitably expressed as the content of sodium ions which is exchangeable by ammonium chloride. Typically, the natural clay material comprising sodium bentonite comprises sodium cations in an amount of at least 20 mmol/100 g, determined by ion exchange with ammonium chloride.

The determination of the content of sodium ions can be carried out by a method comprising refluxing the natural clay material with an excess of ammonium chloride in water for a period of 1 hour, followed by filtration, and analysis of the filtrate by inductively coupled plasma optical emission spectrometry (ICP-OES).

In preferred embodiments, the natural clay material comprising sodium bentonite comprises sodium cations in an amount of at least 30 mmol/100 g, even more preferred in an amount of at least 40 mmol/100 g. Generally, the amount of sodium cations is in the range of 20 mmol/100 g to 100 mmol/100 g, preferably in the range of 30 to 90 mmol/100 g.

A higher content of exchangeable sodium ions indicates a higher content of sodium bentonite in the natural clay material. A higher content of sodium bentonite is desirable because it means that a lower amount of non-sodium bentonite materials need to be removed.

For the present invention, the natural clay material comprising sodium bentonite preferably has a swelling volume of 12 ml or more, determined by adding 2.0 g of the natural clay material comprising sodium bentonite to 100 ml of deionized water. Generally, higher swelling volumes indicate a higher content of sodium bentonite in the natural clay material, which is preferred. In some embodiments, the natural clay material comprising sodium bentonite preferably has a swelling volume of 15 ml or more, or 20 ml or more, such as 25 ml or more, or 30 ml or more. The swelling volume generally is 70 ml or less, or 60 ml or less.

The swelling volume is suitably determined visually. For this purpose, a measuring cylinder is filled with 100 ml of deionized water and 2 g of the respective clay material are added to the water in several portions during a period of 30 minutes. 60 minutes after the last portion of clay material has been added, the volume of swollen material in the measuring cylinder can be determined visually.

The natural clay material comprising sodium bentonite used according to the present invention comprises sodium bentonite, as well as other materials, collectively referred to as non-sodium bentonite impurities. Suitably, the natural clay material comprising sodium bentonite comprises sodium bentonite in an amount of 10 to 90% by weight, calculated on the dry weight of the natural clay material. In preferred embodiments, the content of sodium bentonite in the natural clay material is in the range of 30 to 90% by weight, calculated on the dry weight of the natural clay material.

The content of sodium bentonite in the natural clay material can be determined by removing the non-sodium bentonite impurities from the natural clay material by a method wherein the natural clay material is dispersed in water, followed by removal of non-sodium bentonite impurities by centrifugation at 3700 g for a period of 10 minutes, and drying of the remaining aqueous phase.

The content of non-sodium bentonite impurities in the natural clay material suitably is in the range of 10 to 90% by weight, preferably 10 to 60% by weight, calculated on the dry weight of the natural clay material.

In most embodiments, the non-sodium bentonite impurities comprise at least one of feldspar, calcite, mica, quartz, cristobalite, dolomite, and calcium bentonite.

In step i) of the process of the invention aqueous slurry of a natural clay material comprising sodium bentonite is prepared. The aqueous slurry can be prepared by combining the natural clay material and water in a suitable container. The order of addition of natural clay material and water to the container is not critical. It is also possible to introduce water and natural clay material simultaneously in the container. Tap water or water of similar degree of purity is very suitable for use. If so desired, water of higher purity or deionized water can be used. However, from an economic perspective the use of deionized water is less preferred.

Generally, the aqueous slurry contains the natural sodium clay material comprising sodium bentonite in amount of 2 to 20% by weight, calculated on the weight of the aqueous slurry. While the process can also be carried out in embodiments wherein the amount of natural sodium clay material comprising sodium bentonite is below 2% by weight of the aqueous slurry, such embodiments are less attractive from an economic point of view, because overly large volumes of water have to be handled. If the amount of natural sodium clay material comprising sodium bentonite exceeds 20% by in the aqueous slurry, the viscosity of the slurry may become very high, which detracts from handling the aqueous slurry, such as stirring and pumping. In preferred embodiments, the aqueous slurry contains the natural sodium clay material comprising sodium bentonite in amount of 3 to 15% by weight, calculated on the weight of the aqueous slurry.

In most embodiments, shear force is applied to the aqueous slurry prepared in step i) of the process of the invention. Shear force can be applied by means known to the skilled person, for example by mixers, stirrers or dissolvers, or combinations thereof. Application of shear force can reduce the particle size of the slurry and/or lead to a more uniform particle size distribution within the slurry.

In preferred embodiments, at least one dispersant additive is present during preparation of the aqueous slurry of natural clay material comprising sodium bentonite. The presence of a dispersant additive reduces the viscosity of the slurry at a given content of natural clay material comprising sodium bentonite and water. Furthermore, the presence of a dispersant additive can also improve the separation of non-sodium bentonite impurities in step ii) of the process of the invention. Hence, a dispersant additive can improve the efficiency of the process.

Preferably, the dispersant additive comprises at least one of an organic polymer or oligomer and an inorganic phosphate or polyphosphate salt. Suitable organic polymers or oligomers include linear and branched polymers or oligomers having pendant or terminal carboxylic acid groups or salts thereof, phosphoric acid groups or salts thereof, or phosphonate groups. Suitable polymer or oligomer types include polyester and polyacrylate polymers and oligomers. The weight average molecular weight of the polymers or oligomers generally is in the range of 200 to 250000 g/mol, preferably 500 to 50000 g/mol.

If present, the amount of dispersant additive is suitably in the range of 0.5 to 5.0% by weight, calculated on the weight of the natural clay material comprising sodium bentonite. If the dispersant additive is used in a mount below 0.5% by weight, the beneficial effect of the dispersant additive may not be sufficiently achieved.

In some embodiments, the dispersant additive may contain sodium ions. However, the amount of sodium ions introduced with the dispersant additive is always lower than the amount of sodium ions required for sodium activation of calcium bentonite.

In step ii) of the process of the invention, non-sodium bentonite impurities are removed from the aqueous slurry. The non-sodium bentonite impurities are mainly present in the aqueous slurry in the form of solid particles or incompletely swollen materials, which can be separated from the aqueous phase by physical separation processes, which are generally known to the skilled person. Examples of suitable separation processes include sedimentation, decantation, flotation, and centrifugation. It is also possible to combine such processes or to carry them out in succession, if so desired.

The removed non-sodium bentonite impurities include most of the crystalline impurities and low swellable amorphous minerals and low swellable clays as e.g., calcium bentonite. The better swellable sodium bentonite is mainly not removed and stays in the slurry.

As mentioned above, the non-sodium bentonite impurities to be removed typically comprise at least one of feldspar, calcite, mica, quartz, cristobalite, dolomite, and calcium bentonite.

After the separation step, the aqueous slurry is recovered, and water is removed from the aqueous slurry by spray drying in a spray drying apparatus to prepare a solid treated sodium bentonite clay.

If so desired, shear force may be applied to the aqueous slurry prior to removal of water by spray drying. Generally known techniques for applying shear force may be used for this optional treatment step. Examples of suitable ways to apply shear force include treatment with a high-speed dissolver or passing the slurry through an orifice under pressure.

In step iii) of the process of the invention water is removed from the aqueous slurry by spray drying in a spray drying apparatus to prepare a solid treated sodium bentonite clay.

It has been found that the spray drying is an essential feature of the process of the invention and that drying processes other than spray drying lead to solid treated sodium bentonite clay having inferior properties.

A spray drying apparatus takes a liquid stream and separates the solute or suspension as a solid and the solvent into a vapor. The solid is usually collected in a drum or cyclone. The liquid input stream is sprayed through a nozzle into a hot vapor stream and vaporized. Solids form as moisture quickly leaves the droplets. A nozzle is usually used to make the droplets as small as possible, maximizing heat transfer and the rate of water vaporization. Droplet sizes generally range from 20 to 180 μm depending on the nozzle. There are two main types of nozzles: high pressure single fluid nozzle (50 to 300 bars) and two-fluid nozzles: one fluid is the liquid to dry and the second is compressed gas (generally air at 1 to 7 bars). Instead of atomizing the liquid using a nozzle, a rotary atomizer can be used as well. Rotary atomizers work on the principle of centrifugal energy; this energy is used to produce a high relative speed between the fluid and air which is essential for atomization. A rotary atomizer comprises a rotating surface. This surface can be in the form of a flat or a vaned disc, a cup, or a slotted wheel. The liquid first flows radially outwards in the disc and is then released from the disc's outer limits at a relatively very high speed. The atomization relies on the liquid's flow rate and the disc's rotational speed.

Spray dryers can dry a product very quickly compared to other methods of drying. They also turn a solution (or slurry) into a dried powder in a single step, which simplifies the process.

The inlet air temperature of the spray drying apparatus generally is in the range of 150 to 600° C., preferably in the range of 200 to 450° C.

The spray drying step leads to a removal of water and provides solids particles of treated sodium bentonite clay. Generally, the treated sodium bentonite clay still contains residual amounts of water, for example 20.0% by weight or less, calculated in the total weight of the treated sodium bentonite clay. Preferably, the water content is in the range of 0.1 to 18.0% by weight, more preferably in the range of 0.5 to 15.0% by weight, calculated in the total weight of the treated sodium bentonite clay.

The invention also relates to treated sodium bentonite clay, which is obtainable by or obtained by the process of the invention.

The treated sodium bentonite clay is present in the form of particles. The particles typically have shape wherein all three dimensions have a similar order of magnitude, as opposed to needle- or platelet-like shapes, wherein one or two of the dimensions significantly exceed the other dimension. Generally, the length, width, and height of the particles differ less than 35% of each other.

Suitably, the particles have a d50 number average particle size in the range of 5 to 60 μm, preferably 8 to 40 μm, determined by laser diffraction.

The particles typically have a morel-like structure. This means that the particles have an irregular surface characterized by a network of ridges.

The treated sodium bentonite clay obtainable by the process of the invention suitably has a content of crystalline impurities of less than 10% by weight, preferably less than 5% by weight, and more preferably less than 3% by weight.

The treated sodium bentonite clay has a content of exchangeable sodium ions in an amount of 100 mmol/100 g or less, and a content of exchangeable calcium ions in an amount of 18 mmol/100 g or less, calculated on the dry weight of the clay.

The amount of exchangeable sodium ions is preferably in the range of 50 to 100 mmol/100 g of the treated sodium bentonite, even more preferred in the range of 50 to 90 mmol/100 g.

The amount of exchangeable calcium ions is preferably in the range of 2 to 18 mmol/100 g of the treated sodium bentonite, even more preferred in the range of 2 to 15 mmol/100 g.

The amount of exchangeable sodium ions and calcium ions is suitably determined by a method comprising refluxing the treated clay material with an excess of ammonium chloride in water for a period of 1 hour, followed by filtration, and analysis of the filtrate by inductively coupled plasma optical emission spectrometry (ICP-OES). An excess of ammonium chloride means that ammonium chloride is used in an amount higher than exchangeable ions present in the treated clay material. Suitably, 120 mg of the treated clay material are refluxed with 8 ml of an aqueous solution of ammonium chloride of a concentration of 2 mol/1.

The treated sodium bentonite clay obtainable by the process of the invention is highly suitable for controlling the rheology of an aqueous composition. In particular, the treated sodium bentonite clay can be readily dispersed in numerous aqueous compositions and causes desirable rheological effects. Therefore, the invention also relates to the use of the treated sodium bentonite clay for controlling the rheology of an aqueous composition.

The invention further relates to a method of controlling the rheology of an aqueous composition, comprising adding the treated sodium bentonite clay obtainable by the process of the invention to an aqueous composition.

In the above-mentioned use or method, the treated sodium bentonite clay is suitably added to the aqueous composition in an amount in the range of 0.1 to 7.0%, preferably 0.1 to 5.0% by weight, calculated on the total weight of the aqueous composition.

When the treated sodium bentonite clay is added to an aqueous composition, the viscosity of the aqueous composition generally increases. A higher amount of treated sodium bentonite clay generally causes a higher increase of the viscosity. In some embodiments, the addition of the treated sodium bentonite clay causes a thixotropic behavior of the aqueous composition.

The aqueous composition can be any liquid aqueous composition of which the viscosity should be increased or which should be rendered thixotropic. Aqueous compositions are those in which the main or only liquid diluent used is water. Preferably, aqueous compositions contain less than 35% by weight, 25% by weight, 20% by weight or even less than 10% by weight of (volatile) organic solvents, based on the total weight of water and organic solvent in the liquid formulation. In some embodiments, aqueous compositions are free of organic solvents. Aqueous compositions may contain water-soluble organic or inorganic compounds, e.g., ionic compounds like salts.

Examples of suitable aqueous liquid compositions include a coating composition, a (pre-) polymer composition, a pigment concentrate, a ceramic product, a sealant, a cosmetic preparation, an adhesive, a casting compound, a lubricant, an ink, a cleaning agent, a liquid for use in gas- or oil production, a putty, a metal working fluid, a sprayable liquid, like deposition aids used for crop protection, a wax emulsion, a liquid for use in energy storage media like batteries, a liquid for use in electric or electronic components, a casting or potting composition, and a building material.

The aqueous compositions which are coating compositions or inks can be used in various application fields, like automotive coatings, construction coatings, protective coatings (like marine or bridge coatings), can and coil coatings, wood and furniture coatings, industrial coatings, plastics coatings, wire enamels, foods and seeds coatings, leather coatings (both for natural and artificial leather), color resists (as used for LC displays). Coating materials include pasty materials which typically have a high content of solids and a low content of liquid components, e.g., pigment pastes or effect pigment pastes (using pigments based on aluminum, silver, brass, zinc, copper, bronzes like gold bronze, iron oxide-aluminum); other examples of effect pigments are interference pigments and pearlescent pigments like metal oxide-mica pigments, bismuth oxide chloride or basic lead carbonate.

The cosmetic compositions can be all kind of aqueous liquid compositions used for personal care and health care purpose. Examples are lotions, creams, pastes like toothpaste, foams like shaving foam, gels like shaving gel and shower gel, pharmaceutical compounds in gel like delivery form, hair shampoo, liquid soap, nail varnish, lipstick, and hair tinting lotions.

Preferred wax emulsions are aqueous dispersions of wax particles formed of waxes which are solid at room temperature.

Spraying agents (preferably used as deposition aids) can be equipped with the treated sodium bentonite of the invention in order to achieve drift reduction. They may for example contain fertilizers or herbicides, fungicides, and other pesticides.

The formulations used for construction purpose can be materials which are liquid or pasty during handling and processing; these aqueous materials are used in the construction industry and they become solid after setting time, e.g., hydraulic binders like concrete, cement, mortar/plaster, tile adhesives, and gypsum.

Metal working fluids are aqueous compositions used for the treatment of metal and metal parts. Examples are cutting fluids, drilling fluids (used for metal drilling), mold release agents (mostly aqueous emulsions, e.g., in aluminum die casting and foundry applications), foundry washes, foundry coatings, as well as liquids used for the surface treatment of metals (like surface finishing, surface cleaning and galvanization).

Lubricants are aqueous compounds used for lubricating purpose, i.e., used to reduce abrasion and friction loss or to improve cooling, force transmission, vibration damping, sealing effects, and corrosion protection.

Liquid formulations used for gas and oil production are aqueous formulations used to develop and exploit a deposit. Aqueous drilling fluids or “drilling muds” are preferred examples. An application example is hydraulic fracturing.

Cleaners can be used for cleaning different kinds of objects. They support the removal of contaminations, residual dirt and attached debris. Cleaners also include detergents (especially for cleaning textiles, their precursors and leather), cleansers and polishes, laundry formulations, fabric softeners, and personal care products.

Preferred aqueous compositions include an aqueous coating composition, an aqueous composition comprising a hydraulic binder, an aqueous cleaning composition, and an aqueous personal care composition.

The above-mentioned aqueous compositions may comprise other ingredients and additives commonly used in aqueous compositions, for example organic co-solvents, crosslinkers, anti-foaming agents, dispersing aids, and UV stabilizers. Although the treated sodium bentonite according to the invention provides excellent thickening properties, it is possible to use it in combination with other rheology control agents, if so desired.

Examples of other rheology control agents include polysaccharides (like cellulose derivatives, guar, xanthan), urea compounds, (poly)amides, polyacrylates (like alkali soluble or swellable emulsions), or associative thickeners (like polyurethane thickeners, aminoplast based thickeners, hydrophobically modified alkali soluble emulsion type thickeners).

The treated sodium bentonite of the invention can also be used adsorption agent in certain compositions, for example to absorb undesired impurities.

### EXAMPLES

### Example 1

A natural occurring sodium bentonite raw clay was commercially purchased as “natural sodium bentonite powder”, free of soda ash, with a moisture content of 8% by weight and a montmorillonite content of above 65% by weight. The analyzed swelling volume in deionized water was 19 ml/2 g. The crystalline impurities were analyzed by powder x-ray diffraction. The amount of crystalline impurities was 12% by weight. 250 kg natural sodium bentonite powder were slurried in 4800 kg tap water under vigorous stirring by the use of propeller mixers and a sawtooth dissolver disk for 30 minutes. This slurry was then purified by removing the crystalline impurities and the non-swollen mineral parts by running it over a Flottweg decanter centrifuge with a supplied centrifugal force of approx. 3700 G. About ⅓ of the initial clay amount mass was removed by this decanter centrifuging process. The removed material includes most of the crystalline impurities and low swellable amorphous minerals and low swellable clays as e.g., calcium bentonite. The better swellable sodium bentonite is mainly not removed and stays in the slurry. The overall crystalline impurities were reduced to about 2% by this processing.

For perfection of dispersion, the resulting slurry was additionally run over a Manton-Gaulin homogenizer at a pressure of 150 bar.

The resulting slurry was dried in an Anhydro spray dryer with an inlet drying air temperature of 380° C. The spray feed rate was adjusted to achieve a moisture content in the resulting dried powder of 6% by weight. The spray dryer outlet temperature was in the range of 70° C. to 100° C. The resulting powder had a number average d50 particle size of 15 μm. The treated sodium bentonite had an amount of exchangeable sodium ions of 61 mmol/100 g, and an amount of exchangeable calcium ions of 11 mmol/100 g, calculated on the dry weight of the material.

### Example 2

400 kg natural sodium bentonite powder were slurried in a mixture of 3600 kg tap water and 12 kg BYK-155/35 polyacrylate dispersant. For further treatment the same procedure as in Example 1 was followed. The treated sodium bentonite had an amount of exchangeable sodium ions of 83 mmol/100 g, and an amount of exchangeable calcium ions of 12 mmol/100 g, calculated on the dry weight of the material.

### Example 3

400 kg natural sodium bentonite powder were slurried in a mix of 3600 kg tap water and 4 kg Na-pyrophosphate dispersant. For further treatment the same procedure as in Example 1 was followed.

### Comparative Example 1

Natural sodium bentonite powder, as supplied from mine, without purification, was ground to the same fineness and with same moisture as Example 1. The treated sodium bentonite had an amount of exchangeable sodium ions of 72 mmol/100 g, and an amount of exchangeable calcium ions of 46 mmol/100 g, calculated on the dry weight of the material.

### Comparative Example 2

The purified and homogenized slurry from Example 1 (before spray drying) was lab dried in a lab drying oven at 70° C. to below 12% moisture and ground in a lab mill to the same fineness as Example 1.

### Comparative Example 3

The purified and homogenized slurry from Example 1 (before spray drying) was dried in a drum dryer to below 12% moisture and ground in a lab mill to the same fineness as Example 1.

### Comparative Example 4

Optigel CK, commercially available product from BYK-Chemie GmbH: an artificial sodium bentonite made via alkaline soda activation of a calcium bentonite was processed in the same way as Example 1. The treated sodium bentonite had an amount of exchangeable sodium ions of 135 mmol/100 g, and an amount of exchangeable calcium ions of 20 mmol/100 g, calculated on the dry weight of the material.

### Test Results

### Viscosity in Water

A suspension was prepared by adding the clay (3.5% or 5%, calculated on clay dry weight) to deionized water at room temperature in a glass beaker with a diameter of 70 mm to have in total 200 g water and clay. The addition was done slowly under stirring with a Pendraulik 4 cm diameter toothed cowles disk mounted to a Pendraulik lab stirrer LD 50 at a mixing speed of 930 rpm. When fully added, the dissolver speed was increased to 2800 rpm for a dispersion time of 10 min. After dispersing, the viscosity was measured in a Brookfield DV II at 10 rpm. The value was read after 2 minutes of measuring time. Then the glass beaker was covered and stored at room temperature for the indicated storage times (e.g. 1 hour, 1 day, 1 week) and was measured again.

TABLE 1

| | immediately | 1 h | 1 day | 1 week |
|---|---|---|---|---|
| | Viscosity 3.5% in water | | | |
| | (Brookfield, 10 rpm, mPas), | | | |
| | measured after swelling time | | | |
| Example 1 | 2420 | 3370 | 3560 | 3680 |
| Comparative Example 3 | | 450 | 1270 | |
| Comparative Example 2 | 24 | 280 | 1290 | 2160 |
| Comparative Example 1 | | 4 | 12 | |
| | Viscosity 5.0% in Water | | | |
| | (Brookfield, 10 rpm, mPas) | | | |
| | measured after swelling time | | | |
| Example 1 | 13600 | 13700 | 12400 | 13500 |
| Comparative Example 3 | 1520 | 2670 | 5860 | 7420 |
| Comparative Example 2 | 350 | 1970 | 4130 | 6140 |

The results in Table 1 demonstrate that the thickening effect of the treated sodium bentonite according to the invention ensues much faster than the thickening effect of the comparative sodium bentonites.

### Alpina-Weiss Paint

100 g Alpinaweiß Innenfarbe “Das Original” (flat emulsion paint, DAW SE) and 0.2 g BYK-035 and either 24.43 g of the 3.5% aqueous formulation described in Table 1 (pre-gel) or 17.0 g of the 5% aqueous formulation described in Table 1 (pre-gel)+6.8 g deionized water were added to a Delbrouck beaker no. 211. This was mixed for 5 min. at 2800 rpm with a toothed cowles disk of 4 cm diameter in a Pendraulik lab stirrer LD 50. Then it was covered and stored for 1 day at room temperature. The viscosity was measured in a Brookfield DV II at 10 rpm. The value was read after 2 min. measuring time.

TABLE 2

| | Alpina-Weiss |
|---|---|
| | Paint (10 rpm, |
| | mPas), Pre-gel |
| | (5%) Addition |
| Example 1 (Na-bentonite, spray dried) | 29100 |
| Comparative Example 2 (Na-bentonite, dried in | 23900 |
| lab oven at 70° C.) | |
| Example 3 (Na-bentonite with TSSP dispersant, | 25500 |
| spray dried) | |
| soda activated Turkish white Ca-bentonite, spray dried | 9820 |
| soda activated Turkish white Ca-bentonite, spray dried | 8760 |
| soda activated Moroccan Ca-bentonite, spray dried | 12600 |
| Comparative Example 4 (soda activated grey turkish | 9820 |
| Ca-bentonite, spray dried) | |
| Optigel CK (soda activated grey Turkish Ca-bentonite, | 5960 |
| non-purified, hammer mill grind-dryer) | |
| | Alpina-Weiss |
| | Paint (10 rpm, |
| | mPas), Pre-gel |
| | (3.5%) Addition |
| Example 1 (Na-bentonite, spray dried) | 29300 |
| Comparative Example 2 (Na-bentonite, dried in lab | 24500 |
| oven at 70° C.) | |
| Comparative Example 1 (Na-bentonite, non-purified, | 12800 |
| sun dried) | |

From Table 2 it can be inferred that the treated sodium bentonite according to the invention provides much better thickening in an aqueous paint than sodium activated Ca-bentonite processed in the same way.

### Determination of the Sag Resistance of a White Paint

A white paint was prepared from the ingredients listed in the Table 3 below

| | |
|---|---|
| | Parts by |
| Ingredient | weight |

White paint based on Styrene-Acrylic dispersion

| | |
|---|---|
| demin. water | 12.10 |
| BYK-199 | 0.80 |
| BYK-018 | 0.30 |
| Parmetol A 26 | 0.20 |
| Tioxide TR-92 | 22.00 |
| Clay Thickener | 0.75 |

Dispermat, 20° C., 30 min., 8500 rpm, 4 cm-diameter toothed plate

| | |
|---|---|
| Mowilith LDM 7416 | 63.00 |
| Texanol | 1.60 |

Dispermat, 2 min., 1000 rpm, 4 cm-diameter toothed plate

### Measurement of Sag Resistance

The sag resistance was measured according to ASTM D440-84. This standard test method utilizes a drawdown blade with a series of notches of successively higher clearance. The coating was applied to a test chart with the multi-notch applicator. The charts were immediately hung vertically with the drawdown strips horizontal. The sag resistance is measured from the drawdown after the film has dried completely. The results indicate the maximum layer thickness which can be applied without sagging of the paint.

The results are summarized in Table 4 below:

| Clay Thickener | Sag (μm) |
|---|---|
| Comparative Example 4 | 100 |
| Comparative Example 1 | 100 |
| Comparative Example 2 | 200 |
| Example 1 | 250 |
| Example 3 | 300 |

From Table 4 it can be concluded that the treated sodium bentonite clays according to the invention provide better sag resistance in an aqueous paint than the comparative bentonite clays.

1. A process for treatment of a natural clay material comprising sodium bentonite comprising
i) Preparing an aqueous slurry of a natural clay material comprising sodium bentonite,
ii) Removing non-sodium bentonite impurities from the aqueous slurry, and
iii) Removing water from the aqueous slurry by spray drying in a spray drying apparatus to prepare a solid treated bentonite clay,
wherein the solid treated bentonite clay has a content of exchangeable sodium ions in an amount of 100 mmol/100 g or less, and a content of exchangeable calcium ions in an amount of 18 mmol/100 g or less, calculated on the dry weight of the clay.

2. The process according to claim 1, wherein the natural clay material comprising sodium bentonite has a swelling volume of 12 ml or more, determined by adding 2.0 g of the natural clay material comprising sodium bentonite to 100 ml of deionized water.

3. The process according to claim 1, wherein the non-sodium bentonite impurities comprise at least one of feldspar, calcite, mica, quartz, cristobalite, dolomite, and calcium bentonite.

4. The process according to claim 1, wherein the natural clay material comprising sodium bentonite has a content of non-sodium bentonite impurities in the range of 10 to 90% by weight.

5. The process according to claim 1, wherein shear-force is applied to the aqueous slurry in step i).

6. The process according to claim 1, wherein a dispersant additive is present during preparation of the aqueous slurry of natural clay material comprising sodium bentonite.

7. The process according claim 6, wherein the dispersant additive comprises at least one of an organic polymer and an inorganic phosphate salt.

8. The process according to claim 1, wherein removing non-sodium bentonite impurities from the aqueous slurry is carried out by sedimentation or centrifugation.

9. The process according to claim 1, wherein the spray drying apparatus has an inlet air temperature in the range of 150 to 600° C.

10. The process according to claim 1, wherein the solid treated sodium bentonite clay has a residual water content of 20% by weight or less, calculated on the total weight of the solid treated sodium bentonite clay.

11. A treated sodium bentonite clay, obtained by the process according to claim 1.

12. The treated sodium bentonite clay according to claim 11, wherein the treated sodium bentonite clay is present in the form of particles having a morel-like structure.

13. The treated sodium bentonite clay according to claim 12, wherein the particles have a d50 number average particle size in the range of 5 to 60 μm, determined by laser diffraction.

14. (canceled)

15. (canceled)

16. (canceled)

17. A method of controlling the rheology of an aqueous composition, comprising adding the treated sodium bentonite clay according to claim 11 to an aqueous composition.

18. The method of claim 17, wherein the aqueous composition is selected from an aqueous coating composition, an aqueous composition comprising a hydraulic binder, an aqueous cleaning composition, and an aqueous personal care composition.

19. The method of claim 17, wherein the treated sodium bentonite clay is added to the aqueous composition in an amount in the range of 0.1 to 7.0% by weight, calculated on the total weight of the aqueous composition.

20. The method of claim 17, wherein the treated sodium bentonite clay is added to the aqueous composition in an amount in the range of 0.1 to 5.0% by weight, calculated on the total weight of the aqueous composition.

21. The process according to claim 1, wherein the natural clay material comprising sodium bentonite has a content of non-sodium bentonite impurities in the range of 10 to 60% by weight.
